(19) United States
(12) Patent Application Publication
(10) Pub. No.: US 2026/0130977 A1
(43) Pub. Date: May 14, 2026

(54) PREPARATION METHOD FOR ADENOVIRUS P53-LOADED DENDRITIC CELL VACCINE

(71) Applicant: Scinosen (Shenzhen) Gene Industry Development Co. Ltd., Shenzhen (CN)

(72) Inventors: Diosdado Bautista, Shenzhen (CN); Wei Xu, Shenzhen (CN); Aiguo Hu, Shenzhen (CN); Mao Ye, Shenzhen (CN)

(21) Appl. No.: 19/002,625

(22) Filed: Dec. 26, 2024

(30) Foreign Application Priority Data
Nov. 14, 2024 (CN) 202411631162.5

(51) Int. Cl. A61K 39/00; A61P 35/00; C12N 5/00; C12N 5/0784; C12N 15/86

(52) U.S. Cl. CPC A61K 39/001151; A61P 35/00; C12N 5/0037; C12N 5/0639; C12N 15/86; A61K 2039/5154; C12N 2500/32; C12N 2501/22; C12N 2501/2304; C12N 2506/11; C12N 2710/10043

(57) ABSTRACT

The present disclosure belongs to the field of biotechnology, and specifically relates to a preparation method for an adenovirus p53 (Ad-p53)-loaded dendritic cell (DC) vaccine. The present disclosure includes steps of peripheral blood collection and peripheral blood mononuclear cell (PBMC) separation, PBMC sorting, DC activation, Ad-P53-transfected DC and DC vaccine preparation. P53 can be expressed on a surface of DC as a tumor-associated antigen (TAA) through DC purification, specific multiplicity of infection (MOI) and infection modes, and the Ad-P53-transfected DC has obvious antigen presentation effect, which can be used as a vaccine to activate T cells to kill tumors.

### CROSS-REFERENCE TO RELATED APPLICATION

This application claims priority of Chinese Patent Application No. 202411631162.5, filed on Nov. 14, 2024, the entire contents of which are incorporated herein by reference.

### TECHNICAL FIELD

The present disclosure is in the field of biotechnology, and specifically relates to a preparation method for an adenovirus p53 (Ad-p53)-loaded dendritic cell (DC) vaccine.

### BACKGROUND

p53 gene is recognized as “gene protector”, and more than 60% of human tumors are associated with p53 gene mutation. An exogenous tumor suppressor gene (p53 gene) is introduced into a tumor through an adenovirus vector, and the tumor tissue growth is inhibited through a variety of anti-tumor mechanisms, including apoptosis, by-stander effects and multidrug resistance (MDR) gene expression inhibition. Currently, in clinical practice, adenovirus-mediated p53 gene has been applied to treat a variety of malignant tumors including head and neck tumors, lung cancer, liver cancer, breast cancer, brain tumor, ovarian cancer, bladder cancer, and prostate cancer, etc.

DC is the most powerful professional antigen-presenting cell (APC), which can effectively uptake, process and present antigens. Immature DC has a strong ability to migrate and uptake antigens. Mature DC can effectively activate naive T cells and produce effector cells with anti-tumor activity. Anti-tumor T cells in human body can be effectively activated by Ad-p53-loaded DC, and conventional methods are difficult to integrate P53 gene into DC. Therefore, it is urgent to develop a culture method with high purity of DC, which can make DC overexpresses Ad-p53 protein.

### SUMMARY

An objective of the present disclosure is to provide a preparation method for an Ad-p53-loaded DC vaccine. DC finally prepared by this method is capable of overexpressing Ad-p53 protein, and has broad application prospects.

The objective of the present disclosure is achieved by the following technical solutions.

The present disclosure provides a preparation method for an Ad-p53-loaded DC cell vaccine, including the following steps:
(1) collecting peripheral blood and centrifuging the same, separating a plasma layer and removing a cell layer of the plasma layer, centrifuging again the plasma layer, collecting a supernatant of the plasma layer as autologous plasma for later use, mixing the cell layer with the plasma layer removed with a lymphocyte separation solution in a ratio of 2:1, adding same into a centrifuge tube comprising the lymphocyte separation solution for centrifugation, and sucking a buffy coat to collect peripheral blood mononuclear cell (PBMC), and centrifuging and washing the PBMC again;
(2) collecting CD14+ cells and CD14− cells by incubating and sorting the PBMC with CD14 magnetic beads, and performing an adherent culture of the CD14+;
(3) discarding a supernatant of the CD14+ cells after completing the adherent culture in step (2), adding a DC complete medium and the autologous plasma prepared in step (1) for continued culture to obtain DC;
(4) centrifuging the DC prepared in step (3), re-suspending and placing same in a serum-free XVIVO-15 medium including 1 mg/mL Perminede, using Ad-P53 virus to transfect, and continuing to culture; and
(5) re-suspending the DC cultured in step (4) in the normal saline to obtain a DC vaccine.

Further, in step (1), centrifugal parameters are 2300 rpm for 15 min when the peripheral blood is collected and centrifuged, centrifugal parameters are 2700 rpm for 10 min when the plasma layer is centrifuged again, and step of water bath at 56° C. for 30 min is also included before the plasma layer is centrifuged again.

Further, in step (1), a specific separation method for the PBMC is Ficoll density gradient centrifugation, the lymphocyte separation solution is Ficoll, centrifugal parameters are 2000 rpm for 20 min when the mixture is added to the centrifuge tube including the lymphocyte separation solution, and centrifugal parameters are 1500 rpm for 5 min when centrifugation and washing are performed again.

Further, in step (2), the incubation is performed using 80 μL buffer+20 μL CD14 magnetic beads at 2-8° C. for 30 min in the dark, and 1E7 PBMCs are incubated per 5 μL magnetic beads.

Further, in step (3), compositions of the DC complete medium are 50 ng/mL of IL-4 and 100 ng/mL of granulocyte-macrophage-colony stimulating factor (GM-CSF), an added amount of the autologous plasma is 2%, and the continued culture is performed in an incubator with 5% CO₂ at 37° C.

Further, in step (4), centrifugal parameters are 1800 rpm for 5 min, and the serum-free XVIVO-15 medium including 1 mg/mL Perminede includes L-glutamine (L-Gln) and phenol Red.

Further, in step (4), the transfection is performed by adding the Ad-P53 virus at a ratio of 1×10⁶ DC and multiplicity of infection (MOI) of 25000:1, followed by incubation at a room temperature for 120 min and centrifugation at 1500 rpm for 5 min, a supernatant is sucked and disgarded after completing the centrifugation, and a DC complete medium including 5% autologous plasma is added.

Further, in step (5), the re-suspending is performed by re-suspending transfected DC in a 2 mL of 0.9% normal saline at a cell density of 5×10⁶/mL.

The present disclosure also provides an Ad-p53-loaded DC vaccine prepared by the preparation method.

The present disclosure also provides an application of the Ad-p53-loaded DC vaccine in the preparation of an anti-tumour product.

### Advantageous Effects

The conventional way of infecting DC cannot effectively increase the expression of Ad-P53 protein in DC, while in the present disclosure, the infection rate of P53 can be enhanced through DC purification, specific MOI and mode of infection. In addition, the virus infection rate can be improved using adenovirus as a transfection tool, and P53 protein instead of the current general tumor-associated antigen (TAA) technology as the main component of the vaccine has a more obvious antigen presentation function.

### BRIEF DESCRIPTION OF THE DRAWINGS

To explain the technical solutions of examples in the present disclosure or in the prior art more clearly, the accompanying drawings required in the description of the examples are introduced briefly below. Obviously, the drawings in the following description are only some examples of the present disclosure, and other drawings can be obtained according to these drawings without creative efforts for those ordinary skilled in the art.

FIG. 1 is a flow chart showing the preparation of an Ad-p53-loaded DC vaccine according to the present disclosure.

FIG. 2. shows a flow cytometry detection diagram of P53-infected DC.

FIG. 3 is a diagram showing the in vitro killing effect of T cells activated by P53-infected DC.

### DETAILED DESCRIPTION

Description will now be made in detail to various exemplary embodiments of the present disclosure, which is not to be construed as limiting the present disclosure, but rather as a more detailed description of certain aspects, features and embodiments of the present disclosure.

It is to be understood that the terms used herein is for describing particular embodiments only, rather than limiting the present disclosure. In addition, for numerical ranges within this present disclosure, each intervening value between the upper and lower limits of that range is also specifically disclosed. Any stated value or intervening value in a stated range, as well as any other stated value or every smaller range between stated values or intervening values in the range, is also included in the present disclosure. The upper and lower limits of these smaller ranges may independently be included or excluded in the range.

Unless defined otherwise, all technical and scientific terms used herein have the same meaning as commonly understood by those ordinary skilled in the art to which the present disclosure belongs. Although only preferred methods and materials have been described herein, any methods and materials similar or equivalent to those described herein can be used in the implementation or test of the present disclosure. All documents mentioned in this specification are incorporated by reference to disclose and describe the methods and/or materials in connection with the documents. The contents of this specification shall prevail in case of conflict with any incorporated documents.

It is obvious to those skilled in the art that a variety of modifications and changes can be made in the specific embodiments of the specification of the present disclosure without departing from the scope or spirit of the present disclosure. Other embodiments obtained from the specification of the present disclosure are obvious to those skilled in the art. The specification and examples of the present disclosure are intended to be illustrative only.

The terms “comprising”, “including”, “having”, and “containing”, as used herein, are open terms, meaning to include, but are not limited to.

The test methods used in the following implementations, unless otherwise specified, are all commonly used in the art.

The test materials used in the following implementations, unless otherwise specified, are all commonly used in the art.

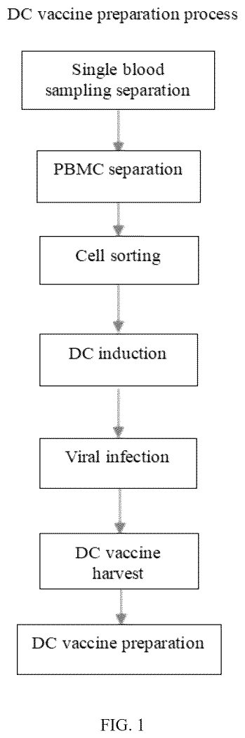

A specific preparation process for an Ad-p53-loaded DC vaccine of the present disclosure is shown in FIG. 1.

### Example 1

A specific preparation method for an Ad-p53-loaded DC vaccine in this example includes the following steps.

### Step 1: Peripheral Blood Collection and PBMC Separation

1.1 Sample centrifugation: a collection tube was used to collect 50 mL of peripheral blood from a tumor patient, a 10 mL disposable sterile pipette was used to transfer all the peripheral blood in the collection tube to a new 50 mL disposable sterile centrifuge tube, and a centrifugation was performed at 2300 rpm for 15 min (acceleration 9 and deceleration 7) at room temperature.

1.2 Plasma separation: an upper plasma layer was carefully sucked using the 10 mL disposable sterile pipette, added in a new 50 mL disposable sterile centrifuge tube, and placed in a water bath at 56° C. for 30 min. The upper plasma layer was inactivated and cooled overnight in a refrigerator at 4° C., and centrifuged at 2700 rpm for 10 min at room temperature. Plasma supernatant was collected into a new 50 mL disposable sterile centrifuge tube using the 10 mL disposable sterile pipette for later use as autologous plasma, and stored in a refrigerator at 4° C.

1.3 PBMC separation: Ficoll density gradient centrifugation was used for separation, and a blood cell layer with plasma removed was mixed with Ficoll at a volume ratio of 2:1, and a mixture was diluted to 30 mL with normal saline using the 10 mL disposable sterile pipette. 15 mL lymphocyte separation solution was added to a 50 mL centrifuge tube using the 10 mL disposable sterile pipette, and the lymphocyte separation solution was purchased from Tianjin MD Pacific Technology Co. Ltd. The diluted blood cells were evenly mixed using a new 10 mL disposable sterile pipette and added into the centrifuge tube (it was to be noted that the blood cells were added very slowly at the beginning above the liquid level, and the blood cells were carefully added above Ficoll's liquid level without damaging a contact surface between the separation solution and the cell suspension), and centrifuged at 2000 rpm for 20 min (acceleration 0 and deceleration 0) at room temperature.

1.4 PBMC collection: after completing the centrifugation, the 10 mL sterile disposable pipette was used to suck and discard liquid in the uppermost layer, and a buffy coat was carefully sucked to collect PBMCs, which were transferred to a new 50 mL disposable sterile centrifuge tube. Samples were taken and counted, and centrifuged at 1500 rpm for 5 min (acceleration 9 and deceleration 7) at room temperature and washed a second time.

### Step 2: PBMC Sorting

2.1 CD14+ magnetic beads sorting and cell attachment: magnetic activated cell sorting (MACS) Running Buffer was prepared to balance room temperature, and two 200 μL disposable sterile suction heads were added with 80 μL buffer+20 μL CD14 magnetic beads and incubated at 2-8° C. for 30 min in the dark, gently shook every 5 min and mixed once (1E7 PBMCs were incubated per 5 μL magnetic bead, the amount of magnetic beads was adjusted according to the specific number of cells, and a volume ratio of magnetic bead incubation Buffer:magnetic beads was 4:1). A large scale (LS) sorting column was placed on a sorting rack, and a 15 mL disposable sterile centrifuge tube was placed under the sorting column. A 1000 μL sterile cartridge suction head was used to suck 3 mL pre-cooled Buffer to equilibrate sorting column for 1-2 times, and the collected Buffer during equilibration was discarded. After incubation by the CD14 magnetic beads, the cells were re-suspended using 3 mL of Buffer (with gently mixing without blowing), and added into the sorting column (using 3 mL for 0-1E8 total cells; and over 1E8 cells were re-suspended using 6 mL, and divided into two columns on average).

2.2 Collection of CD14+ cells and CD14− cells: filtered cells were CD14− cells, and CD14+ cells in the sorting column were collected and centrifuged at 1800 rpm for 5 min (acceleration 9 and deceleration 9). After completing the centrifugation, a 10 mL pipette was used to suck and discard the supernatant, and 2 mL cytokine-induced killer cell (CIK) complete medium 1 was added using a new 10 mL pipette, with the CIK complete medium being an X-VIVO medium including 50 ng/ml cluster of CD3 monoclonal antibody and 100 ng/interferon (IFN)-γ, the CD3 monoclonal antibody purchased from Zhuhai Baso Cell Science and Technology Co. Ltd., and the IFN-7 purchased from Novus Biologicals. After the cells were mixed well, 20 μL of cells were taken to count and calculate the viability, and CD14− cells were used for culturing the CIK.

2.3 CD14+ cell attachment: the supernatant of collected CD14+ cells was sucked and discarded, a serum-free X-VIVO culture medium including L-Gln and Phenol Red was used to adjust a cell concentration to 1×10⁶ cells/mL, and the cells were placed into a carbon dioxide incubator for adherent culture at 37° C. for 30 min.

### Step 3: DC Activation

3.1 The supernatant was removed by a new 10 mL disposable sterile pipette after the adherent culture was completed, and whether there were any unattached cells was observed under a microscope. If there were, 1 mL of X-VIVO serum-free medium was added using the 1000 μL disposable suction head for washing (it is to be noted that the pipette may not touch a bottle wall, without blowing and beating in a whole process), until no obvious T cell residue was observed by naked eyes.

3.2 The DC complete culture medium was added using the 1000 μL suction head, the components of the DC complete culture medium being 50 ng/mL IL-4 and 100 ng/mL GM-CSF serum-free X-VIVO culture medium and 2% autologous plasma, and the cells added with the DC complete culture medium were placed in the incubator with 5% CO₂ at 37° C. for continued culture after being gently and uniformly shook.

Step 4 Transfection of DC with Ad-P53 from Shenzhen SiBiono Biotechnology Co. Ltd.

4.1 On day 5 of culture, DC were taken for collecting all cell suspension into a 50 mL centrifuge tube, and samples were taken for counting living cells before being centrifuged at 1800 rpm for 5 min.

4.2 After counting, the cells were re-suspended in 1 mL of serum-free XVIVO-15 medium including 1 mg/mL Perminede, and the serum-free X-VIVO medium includes L-Gln and Phenol Red. According to 1×10⁶ DC and MOI of 25000:1, Ad-P53 virus was added. The cells added with Ad-P53 were incubated at room temperature for 120 min, and centrifuged at 1500 rpm for 5 min. After centrifugation, the supernatant was sucked and discarded. A DC complete culture medium including 5% autologous plasma was added for continued culture.

4.3 After continuing to culture for 48 h, the supernatant was discarded, and non-adherent cells were washed with PBS. The DC were digested with mild digestive enzyme, and 1×10⁶ cells were taken for flow cytometry to detect an expression of P53, as shown in Table 1.

TABLE 1
Different types of DC

| | | | Amount | |
|---|---|---|---|---|
| | | Addition | added | |
| | Tube | of | per | |
| Type | number | antibody | tube | Remarks |
| DC | 0 | Without | 0 | Blank tube (re-suspension in |
| | | antibody | | 200 μL PBS) |
| | 1 | FVS780 | 200 μL | FVS780 antibody was diluted with |
| | | | | PBS according to a ratio of |
| | | | | 1:1000, i.e. 1 μL of antibody |
| | | | | was stained with 1 mL PBS for |
| | | | | 30 min, and centrifuged, and |
| | | | | the supernatant was discarded |
| | 1 | Anti-P53 | 2 μL | 200 μL PBS was used to re- |
| | | antibody | | suspend the well-mixed antibody, |
| | | | | the re-suspended cells were |
| | | | | incubated in the dark for 30 min |
| | | | | and washed, and a flow cytometry |
| | | | | detection was performed |

(1) Samples to be tested were numbered, No. 0 was a negative control tube, No. 1 was a sample tube, and sequentially numbering was performed according to the number of samples to be tested.

(2) The samples were centrifuged and the supernatant were discarded, and 200 μL PBS was added to No. 0 tube for cell re-suspension, ready for flow cytometry. At the same time, FVS780 antibody was diluted with PBS according to a ratio of 1:1000, that is, 1 μL antibody was added with 1 mL PBS.

(3) After being centrifuged, the samples to be tested were added with 200 μL of diluted FVS620 antibody and incubated at 4° C. for 30 min in the dark after the supernatant was sucked and discarded.

(4) No. 1 tube was added with 1 mL PBS to re-suspend after completing the incubation, and after centrifugation at 300 g for 5 min, the supernatant was sucked and discarded. Anti-P53 and 2 L well-mixed re-suspended cells with 200 μL PBS were added according to an amount of antibody shown in the above table, and incubated at 4° C. for 30 min in the dark.

(5) The No. 1 tube was re-suspended with 1 mL PBS, and centrifugated at 1500 g for 3 min, and the supernatant was sucked and discarded after completing the centrifugation. The No. 1 tube was re-suspended with 200 μL PBS and ready for flow cytometry detection.

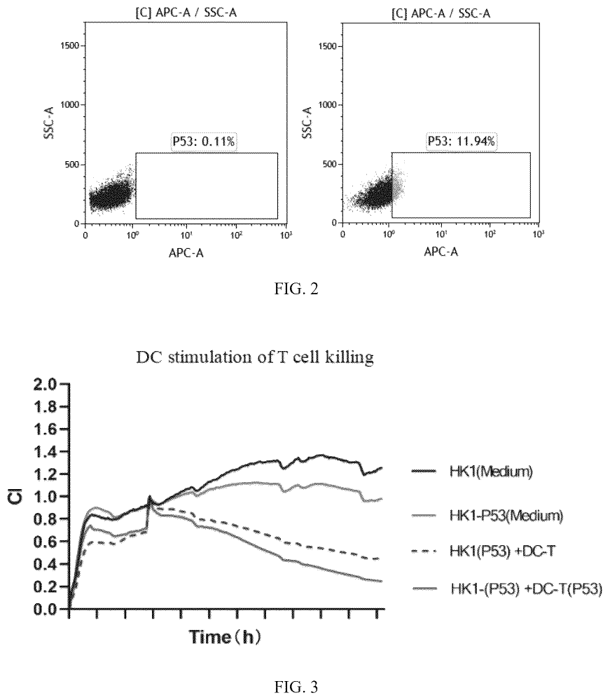

(6) By flow cytometry, an expression of P53 on the surface of mature P53-infected DC was about 11.94%, as shown in FIG. 2.

### Step 5 DC Vaccine Preparation

After DC were harvested and centrifuged, the supernatant was sucked and discarded. The cells were re-suspended in 2 mL of 0.9% normal saline at a cell density of 1×10⁶/mL and mixed well, and 200 μL of cell suspension was taken for endotoxin detection.

Step 6 Co-Culture of P53-Loaded DC with T Cells

6.1 The sorted CD14− cells in step 2.2 were re-suspended in a T cell complete medium, with the T cell complete medium being an X-VIVO medium includign 50 ng/ml CD3 monoclonal antibody and 100 ng/IFN-γ, the CD3 monoclonal antibody purchased from Zhuhai Baso Cell Science and Technology Co. Ltd., and the IFN-γ purchased from Novus Biologicals. The cell suspension was transferred to a corresponding culture container, and incubated in an incubator with 5% CO₂ at 37° C. for continuous culture.

6.2 DC on day 7 of culture were co-cultured with T cells on D7 of culture. After the co-cultured DCT cells were cultured until day 9, HK1 tumor cells were subjected to in vitro killing experiment according to an effector-to-target ratio of 10:1. Finally, it is found that the killing efficiency of T cells activated by the Ad-P53-loaded DC on P53-infected HK1 tumor cells in vitro is higher than that of T cells not activated by P53-loaded DC, as shown in FIG. 3.

The examples described above represent only a few embodiments of the present disclosure and are described in more detail and are not to be construed as limiting the scope of the present disclosure. It is to be noted that several variations and modifications can be made by those ordinary skilled in the art without departing from the concept of the present disclosure, which is within the scope of the present disclosure. Therefore, the protection scope of the present disclosure is to be based on appended claims.

1. A preparation method for an adenovirus p53 (Ad-p53)-loaded dendritic cell (DC) vaccine, comprising the following steps:
(1) collecting peripheral blood and centrifuging the same, separating blood cells and autologous plasma, collecting an upper plasma layer, inactivating the plasma layer and centrifuging the same again, collecting inactivated plasma supernatant as the autologous plasma for later use, diluting cells with the plasma layer removed with normal saline before being mixed well with a lymphocyte separation solution in a ratio of 2:1, adding a mixture into a centrifuge tube comprising the lymphocyte separation solution for centrifugation, and sucking a buffy coat to collect peripheral blood mononuclear cells (PBMC), and centrifuging and washing the PBMC again;
(2) collecting CD14+ cells and CD14− cells by incubating and sorting the PBMC with CD14 magnetic beads, and performing an adherent culture of the CD14+;
(3) discarding a supernatant from the CD14+ cells after completing the adherent culture in step (2), adding a DC complete medium and the autologous plasma prepared in step (1) for continued culture to obtain DC;
(4) centrifuging the DC prepared in step (3) under conditions of 1500 rpm for 5 min, re-suspending and placing same in a serum-free XVIVO-15 medium comprising 1 mg/mL Perminede after completing the centrifugation, using Ad-P53 virus to transfect, and continuing to culture; and
(5) re-suspending the DC cultured in step (4) in the normal saline to obtain a DC vaccine.

2. The preparation method for an Ad-p53-loaded DC vaccine according to claim 1, wherein in step (1), centrifugal parameters are 2300 rpm for 15 min when the peripheral blood is collected and centrifuged, centrifugal parameters are 2700 rpm for 10 min when the plasma layer is centrifuged again, and step of water bath at 56° C. for 30 min is also comprised before the plasma layer is centrifuged again.

3. The preparation method for an Ad-p53-loaded DC vaccine according to claim 1, wherein in step (1), a specific separation method for the PBMC is Ficoll density gradient centrifugation, the lymphocyte separation solution is Ficoll, centrifugal parameters are 2000 rpm for 20 min when the mixture is added to the centrifuge tube comprising the lymphocyte separation solution for centrifugation, and centrifugal parameters are 1500 rpm for 5 min when centrifugation and washing are performed again.

4. The preparation method for an Ad-p53-loaded DC vaccine according to claim 1, wherein in step (2), the incubation is performed using 80 μL buffer+20 μL CD14 magnetic beads at 2-8° C. for 30 min in the dark, and 1E7 PBMCs are incubated per 5 μL magnetic beads.

5. The preparation method for an Ad-p53-loaded DC vaccine according to claim 1, wherein in step (3), compositions of the DC complete medium are 50 ng/mL of IL-4 and 100 ng/mL of granulocyte-macrophage-colony stimulating factor (GM-CSF), an added amount of the autologous plasma is 2%, and the continued culture is performed in an incubator with 5% CO₂ at 37° C.

6. The preparation method for an Ad-p53-loaded DC vaccine according to claim 1, wherein in step (4), centrifugal parameters are 1800 rpm for 5 min, and the serum-free XVIVO-15 medium comprising 1 mg/mL Perminede comprises L-glutamine (L-Gln) and phenol Red.

7. The preparation method for an Ad-p53-loaded DC vaccine according to claim 1, wherein in step (4), the transfection is performed by adding the Ad-P53 virus at a ratio of 1×10⁶ DC, with multiplicity of infection (MOI) of 25000:1, followed by incubation at room temperature for 120 min and centrifugation at 1500 rpm for 5 min, a supernatant is sucked and discharged after completing the centrifugation, and a DC complete medium comprising 5% autologous plasma is added.

8. The preparation method for an Ad-p53-loaded DC vaccine according to claim 1, wherein in step (5), the re-suspending is performed by re-suspending transfected DC in a 2 mL of 0.9% normal saline at a cell density of 5×10⁶/mL.

9. An Ad-p53-loaded DC vaccine prepared by the preparation method according to claim 1.

10. An Ad-p53-loaded DC vaccine prepared by the preparation method according to claim 2.

11. An Ad-p53-loaded DC vaccine prepared by the preparation method according to claim 3.

12. An Ad-p53-loaded DC vaccine prepared by the preparation method according to claim 4.

13. An Ad-p53-loaded DC vaccine prepared by the preparation method according to claim 5.

14. An Ad-p53-loaded DC vaccine prepared by the preparation method according to claim 6.

15. An Ad-p53-loaded DC vaccine prepared by the preparation method according to claim 7.

16. An Ad-p53-loaded DC vaccine prepared by the preparation method according to claim 8.

17. An application of the Ad-p53-loaded DC vaccine according to claim 9 in the preparation of an anti-tumour product.

18. An application of the Ad-p53-loaded DC vaccine according to claim 10 in the preparation of an anti-tumour product.

19. An application of the Ad-p53-loaded DC vaccine according to claim 11 in the preparation of an anti-tumour product.

20. An application of the Ad-p53-loaded DC vaccine according to claim 12 in the preparation of an anti-tumour product.
